(19) United States
(12) Patent Application Publication
(10) Pub. No.: US 2008/0082363 A1
(43) Pub. Date: Apr. 3, 2008

(54) On-line healthcare consultation services system and method of using same

(76) Inventor: Nader Habashi, Annapolis, MD (US)

Correspondence Address: VORYS SATER SEYMOUR PEASE, 1828 L STREET NW, ELEVENTH FLOOR, WASHINGTON, DC, 20036, US

(21) Appl. No.: 11/892,099

(22) Filed: Aug. 20, 2007

Related U.S. Application Data
(63) Continuation-in-part of application No. 11/544,621, filed on Oct. 10, 2006.
(60) Provisional application No. 60/724,272, filed on Oct. 7, 2005.

(51) Int. Cl. A61B 5/00

(52) U.S. Cl. 705/2

(57) ABSTRACT

The present invention relates to a healthcare consultation system that includes a knowledge database, a decision support server, an interactive help system and a connection, wherein the connection comprises a remote assistance unit. The present invention also relates to a wearable computer capable of two-way audio/visual conferencing to provide 24/7 real-time healthcare consultation. The present invention further relates to a method for providing training and help services to a healthcare provider, a healthcare manufacturer or a healthcare user by providing a resource center that comprises a knowledge database, a decision support server and an interactive help system, building the knowledge database by compiling information, analyzing the information in the knowledge database to develop the decision support server, establishing a connection between the interactive help system and the healthcare provider, healthcare manufacturer and healthcare user, accessing the decision support server, and delivering education, training and consultation services via the interactive help system, wherein the connection comprises a remote assistance unit. According to a preferred embodiment, the remote assistance unit comprises a heads-up display, a mini personal computer, a wireless local area network, a headset, a camera, voice over internet protocol technology, a microphone, and/or glasses or head gear.

### CROSS-REFERENCE TO RELATED APPLICATION

This application is a continuation-in-part of U.S. application Ser. No. 11/544,621, filed Oct. 10, 2006, which in turn claims priority from U.S. Provisional Application No. 60/724,272, filed on Oct. 7, 2005, both of which are herein incorporated by reference.

### FIELD OF THE INVENTION

The present invention relates to the field of providing healthcare consultation to human patients. More particularly, the present invention relates to a system and method for remotely connecting healthcare providers and human patients with a multi-disciplinary consulting team of healthcare consultants throughout a geographically dispersed area via a remote assistance unit or robotics and video conferencing systems or other suitable means. The present invention also relates to a wearable computer capable of two-way audio/visual conferencing to provide 24/7 real-time healthcare consultation.

### BACKGROUND OF THE INVENTION

Medicine is evolving through changes in technology and improved methods of delivering healthcare. New technologies and processes enhance the ability of connecting users and suppliers of healthcare.

For example, critical care medicine is undergoing rapid changes due in part to financial limitations, a shortage of intensivists (a physician who specializes in the care and treatment of patients in intensive care units (ICUs)) and nurses, and information overload. Care of critically ill patients consumes a disproportional amount of national medical healthcare dollars (greater than 1% of the GDP). Studies have shown that ICUs with intensivists have reduced ICU costs, hospital costs, mortality rates and lengths of stay in the ICU and the hospital. Moreover, improved patient recovery has been linked to care directed by trained intensivists. Intensivist-directed care has been demonstrated to be superior to traditional care with multiple specialists. However, the current supply of qualified intensivists is inadequate to meet demands. Attempts have been made to address this issue.

U.S. Pat. No. 6,852,107 to Wang, et al., discloses a medical system that allows a mentor to teach a pupil how to use a robotically controlled medical instrument. The system can include a first handle that can be controlled by a mentor to move the medical instrument. The system can further have a second handle that can be moved by a pupil to control the same instrument. Deviations between movement of the handles by the mentor and the pupil can be provided as force feedback to the pupil and mentor handles. The force feedback pushes the pupil's hand to correspond with the mentor's handle movement. The force feedback will also push the mentor's hand to provide information to the mentor on pupil's movements. The mentor is thus able to guide the pupil's hands through force feedback of the pupil handles to teach the pupil how to use the system.

U.S. Pat. No. 6,850,817 to Green discloses a teleoperator system with telepresence that includes right and left hand controllers for control of right and left manipulators through use of a servomechanism that includes a computer. Cameras view workspaces from different angles for production of stereoscopic signal outputs at lines. In response to the camera output a 3-dimensional top-to-bottom inverted image is produced which is reflected by mirror toward the eyes of operator. A virtual image is produced adjacent control arms, which is viewed by the operator looking in the direction of the control arms. By locating the workspace image adjacent the control arms, the operator is provided with a sense that end effectors carried by manipulator arms and control arms are substantially integral. This sense of connection between the control arms and end effectors provide the operator with the sensation of directly controlling the end effectors by hand. By locating the visual display adjacent control arms, the image of the workspace is directly viewable by the operator. Use of the teleoperator system for surgical procedures is also disclosed.

U.S. Pat. No. 6,804,656 to Rosenfeld, et al., discloses a system and method for providing continuous expert network critical care services from a remote location. A plurality of intensive care units (ICUs) with associated patient monitoring instrumentation is connected over a network to a command center which is manned by intensivists 24 hours a day, 7 days a week. The intensivists are prompted to provide critical care by a standardized series of guideline algorithms for treating a variety of critical care conditions. Intensivists monitor the progress of individual patients at remote intensive care units. A smart alarm system provides alarms to the intensivists to alert the intensivists to potential patient problems so that intervention can occur in a timely fashion. A data storage/data warehouse function analyzes individual patient information from a plurality of command centers and provides updated algorithms and critical care support to the command centers.

U.S. Pat. No. 6,356,437 to Mitchell, et al., discloses an apparatus and method for providing a user with task-specific information that includes a portable instruction system that may be worn by a user, and includes, a computer sufficiently lightweight and designed to be worn by a user to which a memory has been connected. The system includes a display device that can receive display signals from the computer for visual display to the user and an input device by which the user enters commands to the computer. An instructional program is provided that the computer accesses and stores in memory in response to a user command and displays information concerning a task to be performed by the user on the display device in response to commands from the user.

U.S. Patent Application Publication No. 2002/0143580 to Bristol, et al. discloses a software-based environment that uses data communication systems or networks, including the Internet and World Wide Web technologies, to implement a dosage calculator, which assists the clinician in managing patients receiving intrathecal therapy. Medical professionals require integrated data to manage their patients. Implantable drug pumps can supply robust data associated with the drug pump. The invention provides for a communications environment for clinicians, pharmacists, drug pump manufactures, and patients to assess not only the information supplied by an implantable drug pump, but also integrate data from other data sources. The invention provides for an inexpensive and practical way for a physician to review the performance parameters of an implantable drug pump in a patient to provide optimization of the life of the drug pump and the therapeutic substance formulations for the patient.

However, there remains a need for a cost-effective process that reduces lengths of stay in a hospital, including in an intensive care unit (ICU), enhances the effectiveness of physicians, especially intensivists, and improves handling of healthcare information. This need will become more relevant as healthcare evolves and there is less on-site expertise, increasing nursing and physician shortages, and an ageing population, combined with the increasing complexity of healthcare treatments and technologies.

### SUMMARY OF THE INVENTION

It is an object of the present invention to provide a system and method of providing access to medical expertise from a healthcare professional or intensivist through broadband, cellular or satellite technology. More preferably, the present invention relates to a system comprising a resource center which comprises a knowledge database, a decision support server, an interactive help system and a connection, wherein the connection comprises a remote assistance unit and a method of using same. According to a preferred embodiment, the resource center is equipped with an intensivist-led multi-disciplinary team.

It is another object of the present invention to provide an interactive help system that provides an interface between a healthcare provider, a healthcare manufacturer and/or healthcare user. More preferably, the present invention relates to a system and method to connect, preferably via an on-line or wireless connection, geographically dispersed patients and clinicians (users and suppliers) with experts, preferably intensivists, located at a dedicated remote resource center. According to a preferred embodiment, the system further comprises healthcare user data.

It is another object of the present invention to provide improved home healthcare, health maintenance programs, extended care facilities, intermediate care units and intensive care units.

It is another object of the present invention to utilize existing patient information systems for the purpose of centralizing and organizing patient information and providing a portal to leverage medical expertise, consulting and patient monitoring from geographically dispersed regions.

It is another object of the present invention to provide an interactive help system comprising at least one of an automated phone system, an intranet system, an internet access or a product provider help menu. According to another preferred embodiment, the system and method includes a remote assistance unit or robotic means for connecting users, suppliers and/or experts.

It is another object of the present invention to provide a remote assistance unit that comprises a heads-up display, a mini personal computer, a wireless local area network, a headset or headphone, a camera, voice over internet protocol technology (VoIP), a microphone and/or glasses or head gear.

It is another object of the present invention to provide a heads-up display that combines a micro display with near-to-the-eye (NTE) technology and a forward-viewing miniature camera, bi-directional microphone and speaker contained in the headgear and connected to an intranet or internet via wireless broadband width or cellular spectrums. More preferably, this system is further connected to a server that provides file sharing and interacts between the wearer of the headgear and a remote location for the purpose of real time audio video conferencing, data exchange and demonstration and instruction.

It is another object of the present invention to provide a system and method that uses a remote-controlled, computerized robot with two-way audiovisual capabilities to provide a plurality of geographically dispersed patients and clinicians access to a remote and dedicated resource center equipped with medical services, expertise, education and training.

It is another object of the present invention to provide medical manufacturers and the healthcare industry access to intensivists at a dedicated resource center via a remote assistance unit or robotics technology for the purpose of supporting customer training, education, trouble-shooting and medical consulting regarding the use of specific technology. In a preferred embodiment, the present invention provides medical expertise to medical manufacturers, the healthcare industry and their respective customers for the purpose of internal staff training, education, clinical application and sales support.

It is a further object of the present invention to provide healthcare providers, medical manufacturers and the healthcare industry access to a clinical resource center through voice data broadband (voice and data transmitted over a TCP/IP network, cellular or satellite) and other enabling technologies such as telephone (land line), cellular phone and email. According to a preferred embodiment, the present invention includes a remote assistance unit or robotics and remote medical services to deliver healthcare and medical consulting services by connecting a user and a provider to such services regardless of their location, including but not limited to any of the following settings: ICU; pre-ICU or monitored/intermediate care units; emergency room or triage area; any emergency situation within a hospital; ambulances; medical evacuation helicopters; airports and airplanes; cruise ships; trains, subways and buses; shopping centers and malls; patient homes; home health emergency services; extended care facilities; home health maintenance services; sales and customer support for medical products and pharmaceuticals; military; and mass casualty and terrorism events.

It is a further object of the present invention to reduce the ICU length of stay by leveraging intensivist expertise over a large geographic area to enhance the effectiveness of intensivists. According to a preferred embodiment, the present invention addresses the supply and demand issue by providing a dedicated service that allows member hospitals, medical industries and healthcare professionals access to intensivists on demand, 24 hours a day, 7 days a week (24/7).

It is a further object of the invention to provide 24/7 access to healthcare consultation via a remote assistance unit, wherein the remote assistance unit is preferable portable. According to a preferred embodiment, a nurse, physician or other healthcare provider connected to the remote assistance unit of the present invention can interact with a resource center staffed by experts who have access to a knowledge database and decision support database, and can assist the nurse, physician or other healthcare provider in various real-time situations, which can vary from troubleshooting alarms on a medical device, assisting in patient care, providing a resource during a procedure or therapy, patient monitoring and management or act a resource for current therapies and treatments and management options.

It is yet another object of the present invention to provide a robot capable of self-powered mobility that is controlled remotely and capable of roaming to any patient care area where there is a wireless access point. According to a preferred embodiment, the robot is capable of providing bi-directional interactive video teleconferencing between the patient care area, preferably a hospital, and remote site(s) for educational or consulting purposes wherein clinicians are capable of accessing a network via broadband technologies from remote locations to access the robot.

It is yet another object of the present invention to enable remote medical services to be offered through a heads-up display or robotics and an on-line system, wherein the services include, but are not limited to, expert consulting services, patient monitoring, training, education and in-services (training sessions). According to a preferred embodiment, patients and hospital staff and healthcare personnel, including but not limited to registered nurses (RNs), respiratory therapists (RTs), respiratory care practitioners (RCP) and medical doctors (MDs), can utilize the enabled medical service.

It is yet another object of the present invention to provide a cost-effective system and method that reduces ICU length of stay, enhances the effectiveness of an intensivist and improves information handling by connecting clinicians with an intensivist-led multi-disciplinary team. According to a preferred embodiment, the present invention is utilized as an element in the integrated delivery system (DS) of a major medical facility or a consortium of hospitals, including any group of healthcare service units that typically includes hospitals, physicians (including, for example, medical groups and independent practice associations), and other non-hospital providers (for example, ambulatory surgery centers, home health providers, skilled nursing facilities, etc.).

There has thus been outlined, rather broadly, the more important features of the invention in order that the detailed description thereof that follows can be better understood, and in order that the present contribution to the art can be better appreciated. There are, of course, additional features of the invention that will be described further hereinafter.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Accordingly, those skilled in the art will appreciate that the conception upon which this disclosure is based can be readily utilized as a basis for the designing of other structures, methods and systems for carrying out the several purposes of the present invention. It is important, therefore, that equivalent constructions, insofar as they do not depart from the spirit and scope of the present invention, are included in the present invention.

For a better understanding of the invention, its operating advantages and the aims attained by its uses, reference should be made to the accompanying drawings and descriptive matter which illustrate preferred embodiments of the invention.

### BRIEF DESCRIPTION OF THE DRAWINGS

FIG. 1 is a schematic illustrating a preferred embodiment of the system of the present invention wherein a resource center is connected to various users;

FIG. 2 is a schematic illustrating a preferred embodiment of the system of the present invention wherein a site requiring medical consultation services is connected to the resource center with various means of communication;

FIG. 3 is a schematic illustrating a preferred embodiment of the system of the present invention wherein the system comprises a feedback loop;

FIG. 4 is a flowchart illustrating a preferred embodiment of the method of the present invention for building a knowledge database;

FIG. 5 is a flowchart illustrating a preferred embodiment of the method of the present invention wherein a resource center provides healthcare services to various users;

FIG. 6 is a flowchart illustrating a preferred embodiment of the method of the present invention as can be used in the event that a caregiver such as a nurse notices a change in patient status;

FIG. 7 is a flowchart illustrating a preferred embodiment of the method of the present invention as can be used in the event that a caregiver such as a physician who is at home requests a consultation;

FIG. 8 is a flowchart illustrating a preferred embodiment of the method of the present invention as can be used in the event that a caregiver such as a physician in an emergency room (ER) requests a consultation;

FIG. 9 is flowchart illustrating a preferred embodiment of the method of the present invention wherein a resource center provides training and help services to healthcare manufacturers; and

FIG. 10 is a schematic illustrating a preferred embodiment of the system of the present invention wherein a remote assistance unit is connected to the resource center with various means of communication.

### DETAILED DESCRIPTION OF PREFERRED EMBODIMENTS

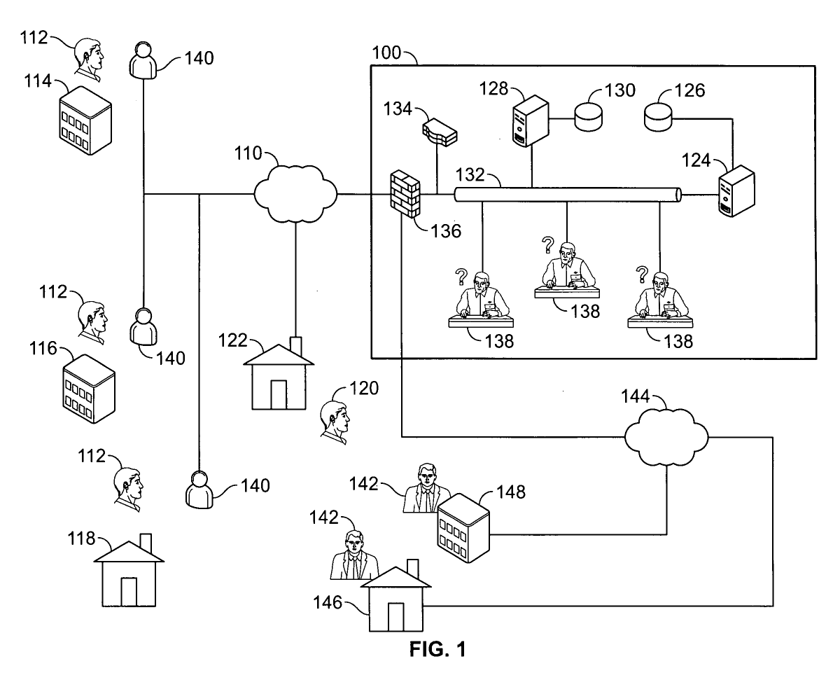

As seen in FIG. 1, the system of the present invention comprises a resource center 100 connected via a means of connection such as Internet/WAN 110 to various healthcare users 112. A healthcare provider 120 is also connected via the Internet/WAN 110 to the resource center 100 and the healthcare users 112. The healthcare user 112 can be a patient or secondary healthcare provider who in turn provides healthcare to a patient. The healthcare provider 120 includes any healthcare professional, such as a nurse, therapist, physician, surgeon, dentist, etc. The healthcare user 112 can be located anywhere such as in an extended care facility 114, in a hospital 116, at home 118, in a clinic (not shown), on an airplane (not shown), on a ship (not shown), on a bus (not shown), on a train (not shown), etc. A healthcare provider 120 can be located anywhere, such as at the office, at home, at another healthcare facility or at another location, collectively 122. A healthcare provider 120 can also be in transit, such in a car, on a ship or in an airport.

According to a preferred embodiment, the resource center 100 is in a location that is remote from the location where the primary care is being provided. Within the context of the present invention, remote means physically distinct, which could be as close as a few feet, e.g., down the hall from the location where the primary care is being provided or as far as thousands of miles away from where the primary care is being provided, e.g., on another continent.

The resource center 100 is connected via an internal TCP/IP network 132 or equivalent means to a decision support server 124 and a knowledge server 128. The network 132 is connected to an internal router 134 that is connected thorough an internal firewall 136 to the Internet/WAN 110. Further, the resource center 100 can be staffed with clinical experts 138.

The decision support server 124 is connected to a decision support database 126. The decision support server 124 is a computer server that operates software application(s), as would be known to one of skill in the art. The decision support database 126 stores the information necessary to support the decision support server 124.

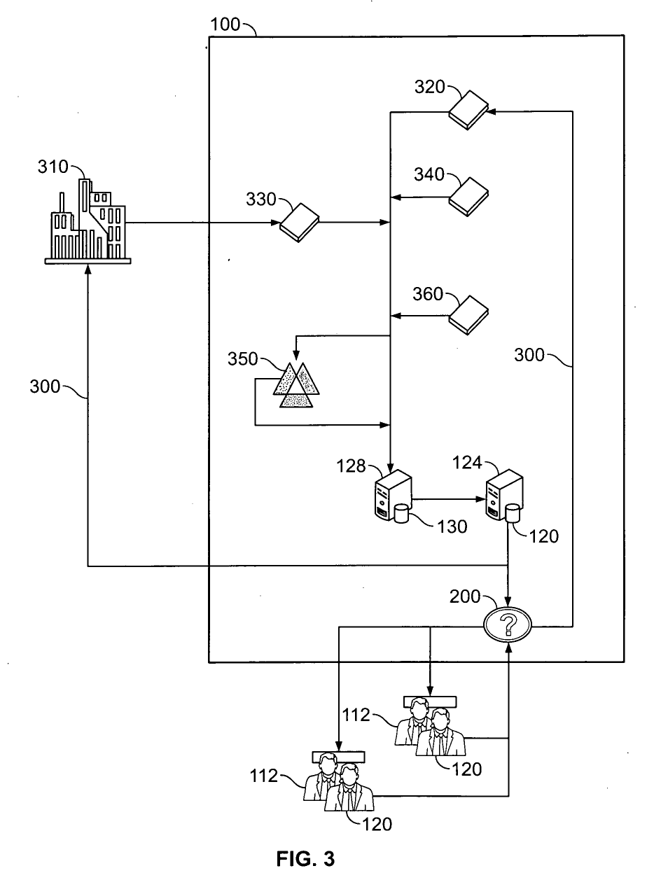

The resource center 100 also comprises a knowledge server 128 and a knowledge database 130. The knowledge database 130 of the present invention comprises a retrievable database that gathers and stores medical information. As can be seen in FIG. 3, the medical information includes, but is not limited to, healthcare manufacturer information 330, medical literature 340, a laboratory 350, clinical information 360, clinical experts 138 and/or specialty consultants 142 (as seen in FIG. 1). More preferably, the healthcare manufacturer information 330 comprises product reference material. Furthermore, the laboratory 350 can comprise a testing laboratory or an analysis laboratory for testing or analysis of healthcare products. The testing laboratory or analysis laboratory can be a testing area that tests and/or reproduces scenarios and interaction with medical devices to increase and enhance the safety and efficacy of a healthcare product such as a medical device or drug.

According to the present invention, the system includes a means for connecting the healthcare users 112 via the Internet/WAN 110 to the resource center 100. Such means can include a remote assistance unit 101 or a deployable robot 140. According to a preferred embodiment, the robot 140 can be a mobile, interactive video conferencing unit remotely controlled by the clinical expert 138 located at the resource center 100 or other locations.

The present invention further includes a system and method for remotely monitoring healthcare users 112, such as a patient, including a system and method for generating and transmitting input commands to a robot 140 from a remote location such as the resource center 100. The resource center 100 can include a personal computer that is operated by a clinical expert 138, such as a doctor. The input commands can move the robot 140 so that a robot camera and microphone can capture the video image and sounds of the patient and transmit them back to the remote resource center 100. The robot 140 can also have a monitor and a speaker to allow for two-way video conferencing between the patient and a doctor at the remote resource center 100. The robot 140 can move from room to room so that a doctor can make “patient rounds” within a medical facility. The system thus allows a doctor to visit patients from a remote location, thereby improving the frequency of visits and the quality of medical care. An example of such a robot is disclosed in U.S. Published Patent Application No. 2004/0143421 to Wang, et al., entitled “Apparatus and method for patient rounding with a remote controlled robot,” the entire disclosure of which is herein incorporated by reference.

According to a preferred embodiment, specialty consultants 142, located in a consultant's home 146, office 148 or elsewhere, are connected to the resource center 100 via an Internet/WAN 144. In one embodiment, Internet/WAN 144 is the same Internet/WAN 110 that connects the resource center 100 to the healthcare user 112. In an alternate embodiment, Internet/WAN 144 is a separate and/or distinct means of connection. The specialty consultants 142 can be specialty healthcare consultants that provide an external source of medical information.

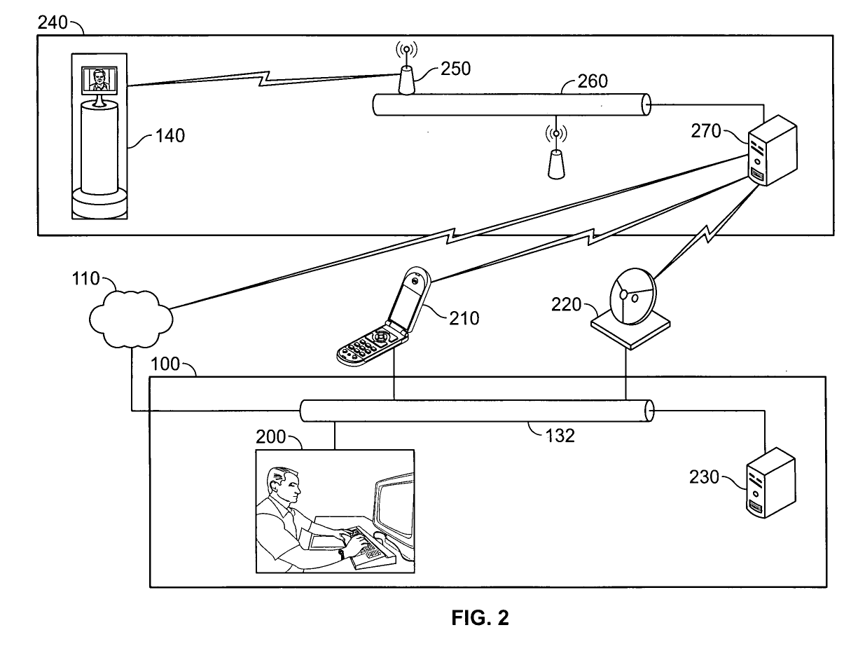

As seen in FIG. 2, the resource center 100 is preferably a dedicated site with multi-communication technologies staffed 24/7 by clinical experts 138 and connected via a connecting means such as the Internet/WAN 110, a cellular/3G connection 210 or a satellite connection 220 to a site requiring medical consulting services 240. According to a preferred embodiment, the resource center 100 is a remote location staffed with clinical experts 138 at an interactive help system 200 or consulting station. The interactive help system 200 has access via the network 132 to an internal communication server 230 that is in turn connected to the knowledge database 130, the decision support server 124, medical devices and scenario reproduction (not shown). More preferably, clinical experts 138 in the interactive help system 200 are equipped with bidirectional interactive voice, data and image transmission capabilities and are available to healthcare users 112 such as patients or secondary healthcare providers located at the site requiring medical consulting services 240. The site requiring medical consultation services 240 is preferably equipped with mobile two-way interactive audio-visual capabilities, such as a remote assistance unit 101 or robot 140 that is connected via a wireless connection 250 to a site TCP/IP network 260 that is in turn connected to a site communication server 270. More preferably, the resource center 100 is a critical care resource center, a rapid response resource center, an emergency care resource center or a mass casualty response center.

According to a preferred embodiment, the site requiring medical consulting services 240 is a hospital 116, specialized transplant center or donor hospital in need of medical expertise to assist in providing support to the healthcare provider 120 that is to procure suitable donor organs. In this case, the transplant center or donor hospital would have access to the resource center 100 and the clinical experts 138 in the resource center 100 would provide the technical expertise to permit the healthcare provider 120 at the hospital 116 or transplant center to procure the organs.

The connection according to a preferred embodiment of the present invention can be a remote connection that connects the clinical experts 138 to healthcare providers 120, healthcare users 112 and healthcare manufacturers 310 located at geographically dispersed locations. The connection can also be a wireless telephone connection, dial-up telephone connection, wireless internet connection, cable connection or DSL connection. The connection can also comprise a telephone system, a facsimile system, an electronic mail system, a video system, a video conferencing system, an intranet system, an internet system, a heads-up display (HUD) 106 or a robot 140. According to a preferred embodiment, the robot 140 can comprise mobile video conferencing, more preferably, a clinical expert 138 located in a remote location such as the resource center 100 can control the robot 140.

Further examples of wireless access points include Cisco®, a device that allows wireless access to a local or wide area network (WAN). Otherwise, a wireless access point can be any device with radio transmitting and receiving capabilities that typically operates using 802.11a, b or g protocol. “802.11” refers to a family of specifications developed by the Institute of Electrical and Electronics Engineers (IEEE) for wireless LAN technology. 802.11 specifies an over-the-air interface between a wireless client/customer and a base station or between two wireless clients/customers.

According to a preferred embodiment, the clinical experts 138 comprise an intensivist-led multi-disciplinary team. The multi-disciplinary team according to the present invention is a group of healthcare professionals with diverse specialties (medical, social, educational, developmental, etc.) who work together to develop an organized approach to the total management of a healthcare user 112 such as an ICU patient.

As seen in FIG. 3, the resource center 100 can also comprise a feedback loop 300, wherein the feedback loop 300 provides feedback to a healthcare manufacturer 310 or to the knowledge database 130, to enhance or improve products such as medical devices, medical protocols and/or drugs. As used herein, a healthcare manufacturer 310 comprises at least one of a medical device manufacturer or a drug manufacturer. More specifically, the step of providing the feedback loop 300 can comprise providing feedback to the healthcare manufacturer 310 via the feedback loop 300, preferably to increase and enhance the safety and efficacy of a healthcare product such as a drug or medical device. The step of providing the feedback loop 300 can also comprise providing feedback to the knowledge database 130 via the feedback loop 300 to develop and build a comprehensive and robust knowledge database 130 as seen in FIG. 3.

According to a preferred embodiment, the resource center 100 can further comprise healthcare user data 320 such as demographic data, pharmacological data, physiological data, radiological images, hemodynamic parameters, laboratory data, device output data, audio data and/or video data. Demographic data includes information about a healthcare user 112, such as the name, race, gender, etc. Pharmacological data includes information on the type, route and amount of pharmaceutical drugs that a healthcare user 112 receives. Radiological images include, but are not limited to, x-rays, CAT scans, MRI, angiography, and the like. Hemodynamic parameters include vital signs such as blood pressure, heart rate, respiratory rate, pulmonary artery pressure, etc. Laboratory data includes results of tests from blood, urine, spinal fluid, etc. as tested for cell count, bacteria, etc. Device output data includes data transmitted from a medical device to an application that displays or stores that data for retrieval. Audio and video data includes data generated from video conferencing with a mobile or stationary video conferencing device that is displayed and can be stored electronically. According to a preferred embodiment, the electronic feed for the healthcare user data 320 is available in real time, i.e., provides present time healthcare user data 320.

According to a preferred embodiment, the system of the present invention can also integrate data and information from other devices such as medical devices. For example, the system of the present invention can integrate data from patient monitors, continuous renal replacement therapy (CRRT) devices, ventilators and other data systems such as patient data systems or data from bar scanners. Preferably, this information can be selected and customized for display. According to another embodiment, the system of the present invention is also capable of displaying alarms from medical devices and can use the decision support database 126 to aid in increasing patient safety. For example, before medications are given and by using bar scanning technology, the system of the present invention would track the medication to be given and would query a database to review allergies, drug interactions, validate patient identity and all available patient data to alert the healthcare provider 120 of any precautions that need to be taken.

Healthcare user data 320 can be stored in information systems known by those of ordinary skill in the art. Such systems include, but are not limited to, systems made by Cerner, iMD Soft, CliniComp, Drager, GE, Phillips, GCQ, Eclipsys and Piscis. Access to such information systems provides access to critical patient information when decisions need to be made.

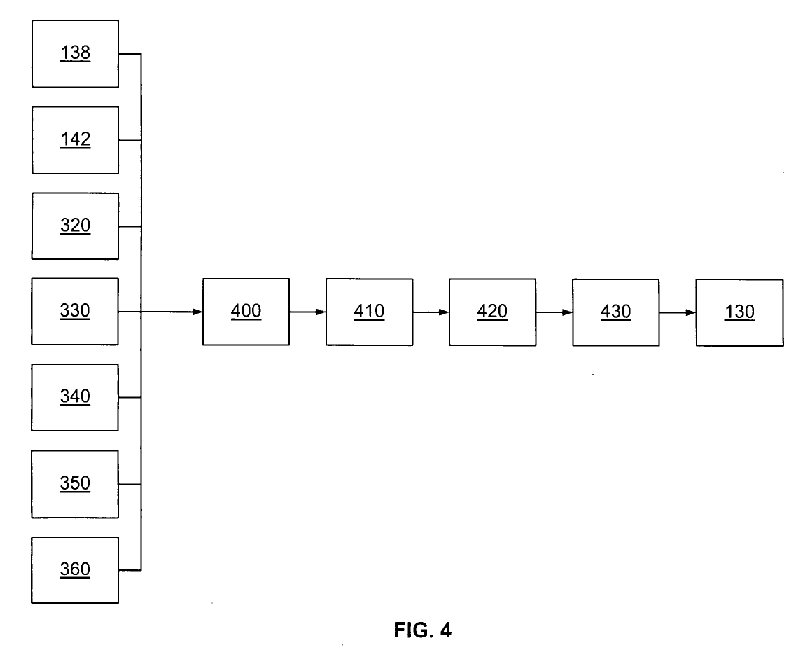

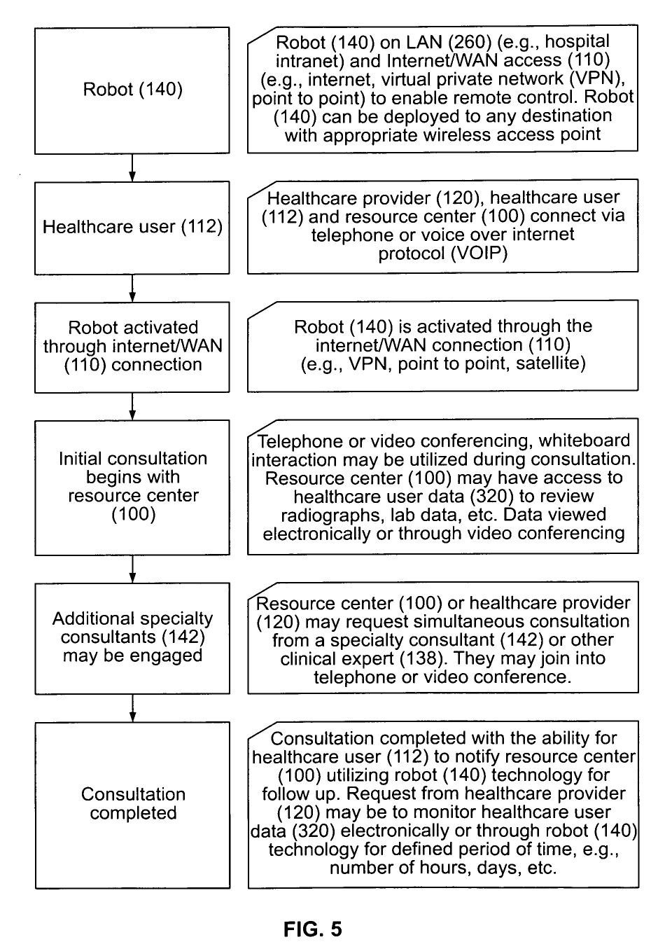

According to another preferred embodiment, the step of building the knowledge database 130 comprises the step of collecting information and data 400 from clinical experts 138, specialty consultants 142, healthcare user data 320, healthcare manufacturer information 330, medical literature 340, a laboratory 350 and/or clinical information 360 and the step of processing same 410 as seen in FIG. 4. Once the information and data is collected 400 and processed 410, a document is drafted 420 and then validated 430 for content accuracy and quality assurance.

Where needed, there are several existing data warehousing technologies known by those of ordinary skill in the art. Examples of such technologies include, but are not limited to Oracle data warehouse, Microsoft SQL server, IBM DB2 data warehousing and Cognos and Brio data warehouse tools.

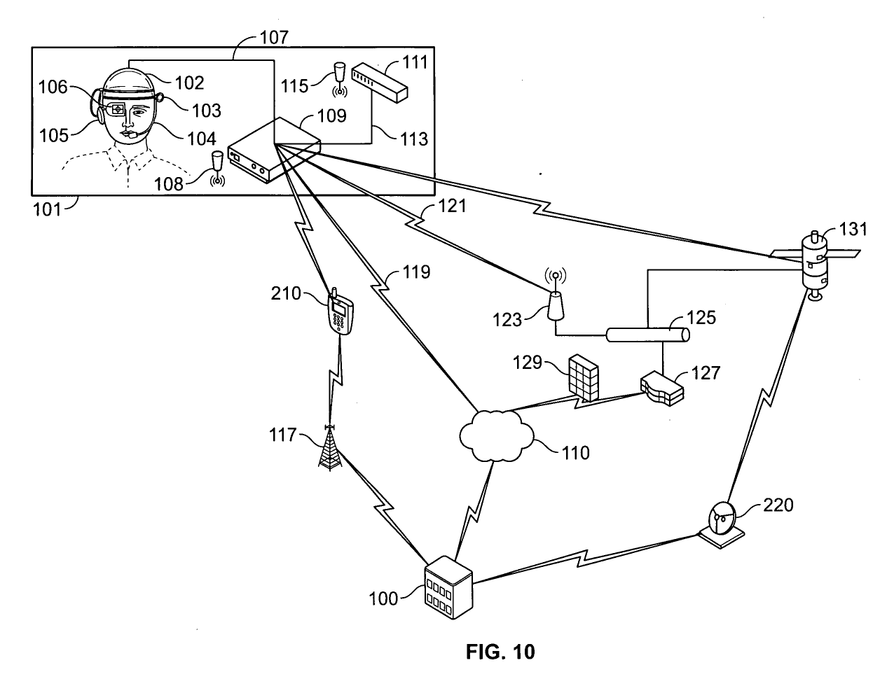

The present invention also relates to a method for healthcare manufacturers to provide training and help services to healthcare providers 120 or healthcare manufacturer employees comprising the step of establishing a connection between a medical expert and the healthcare provider 120 or healthcare manufacturer employee, wherein the medical expert provides education and training services. Preferably, the present invention relates to a method for providing training and help services to a healthcare provider 120, a healthcare manufacturer 310 or a healthcare user 112 comprising the steps of providing a resource center 100 that comprises a knowledge database 130, a decision support server 124 and an interactive help system 200, building the knowledge database 130 by compiling information 400, analyzing the information 410 in the knowledge database 130 to develop the decision support server 124, establishing a connection 110 between the interactive help system 200 and the healthcare provider 120, healthcare manufacturer 310 and healthcare user 112, accessing the decision support server 124, and delivering education, training and consultation services via the interactive help system 200 wherein the connection comprises a remote assistance unit 101 as illustrated in FIG. 10, wherein the step of building the knowledge database 130 comprises compiling information from at least one of healthcare manufacturer information 330, medical literature 340, reference material, a laboratory 350, clinical information 360 or an expert clinical consultant. More preferably, the method can further comprise the step of collecting healthcare user data 320. According to a preferred embodiment, the method further comprises the step of providing feedback to the healthcare manufacturer 310 or the knowledge database 130 via a feedback loop 300.

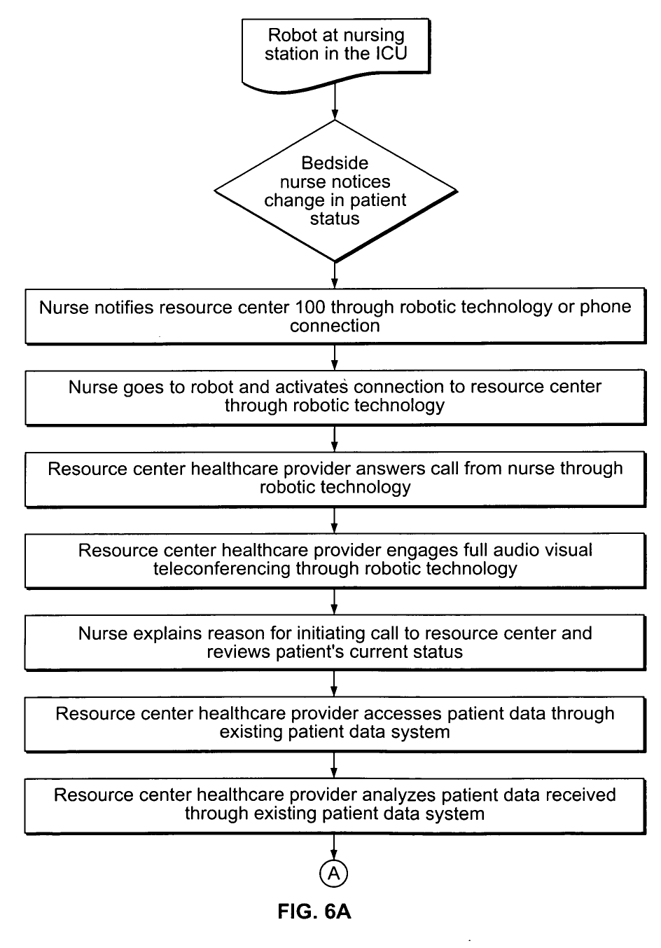

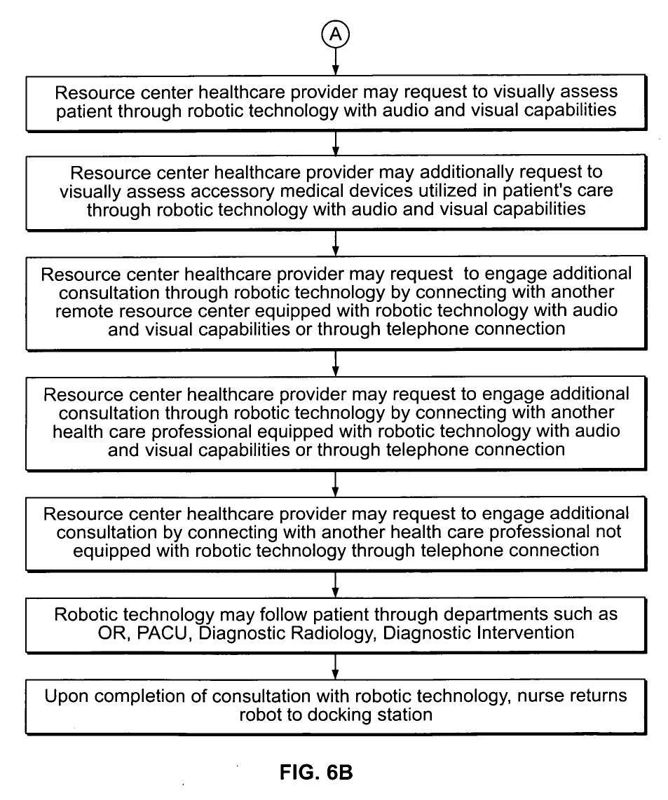

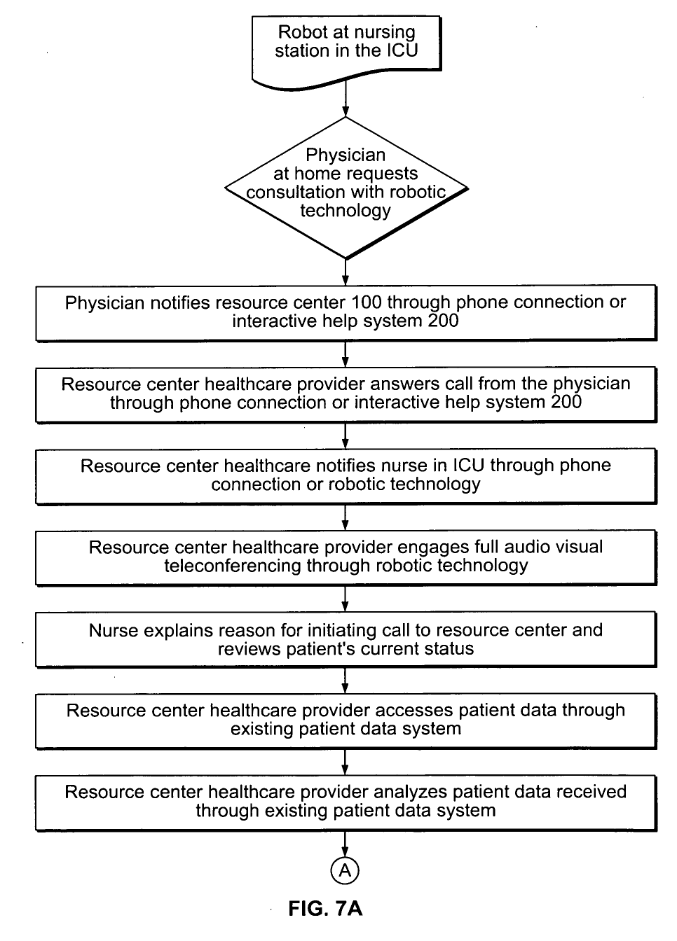

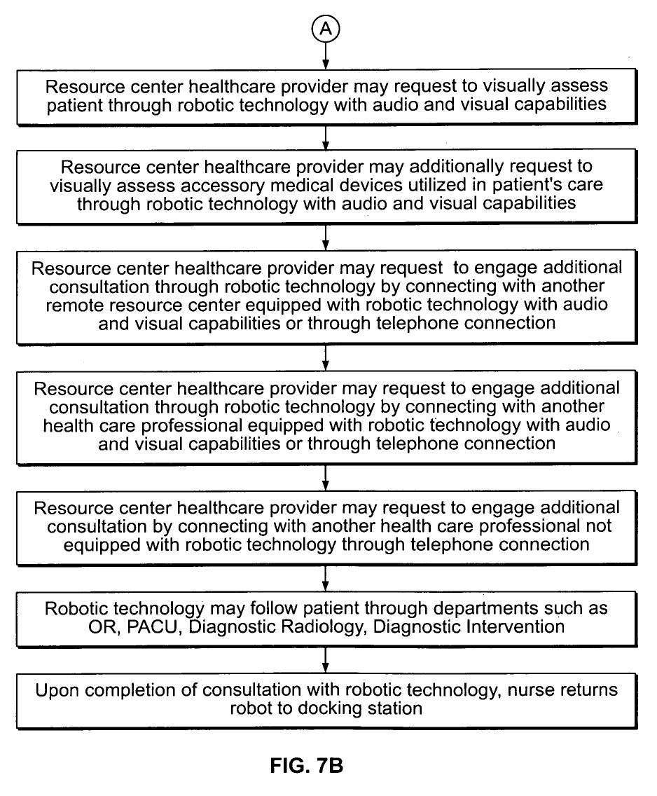

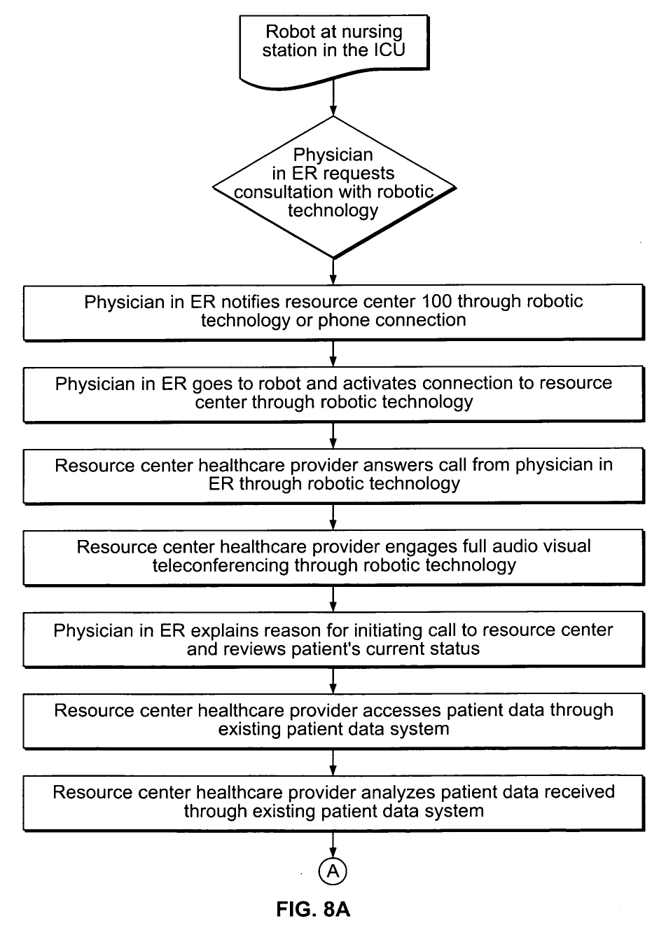

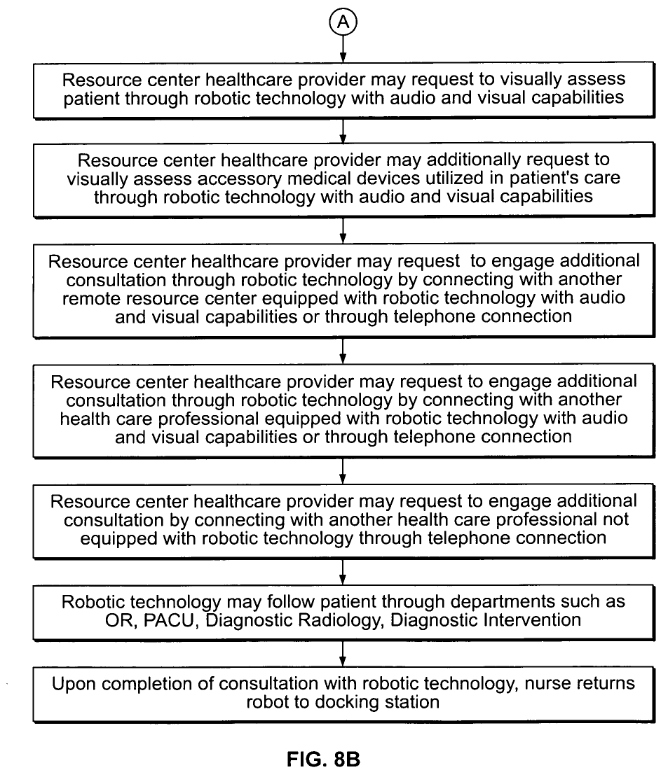

According to another preferred embodiment, the delivering step further comprises the step of delivering education, training and consultation services 24/7. The consultation services can include medical illness, medical device, educational, policy and procedure, ICU evaluation and assessment, staffing, protocol development and decision support and medical services, including corporate medical services. In one example the invention can be used to connect remote clinical experts 138 to healthcare providers 120 to enable real-time assistance with active patient issues. More specifically, the system and method of the present invention can be used in the event that a caregiver such as a nurse notices a change in patient status as shown in FIG. 6; in the event that a caregiver such as a physician who is at home requests a consultation as shown in FIG. 7; or also in the event that a caregiver such as a physician in an emergency room (ER) requests a consultation as shown in FIG. 8.

According to another preferred embodiment, the consultation could be initiated by a request from a healthcare provider 120 to monitor a healthcare user 112. In this case, the resource center 100, located in a location remote from the healthcare user 112, would notify the healthcare provider 120 in the event of any noted anomaly that may affect the healthcare user 112.

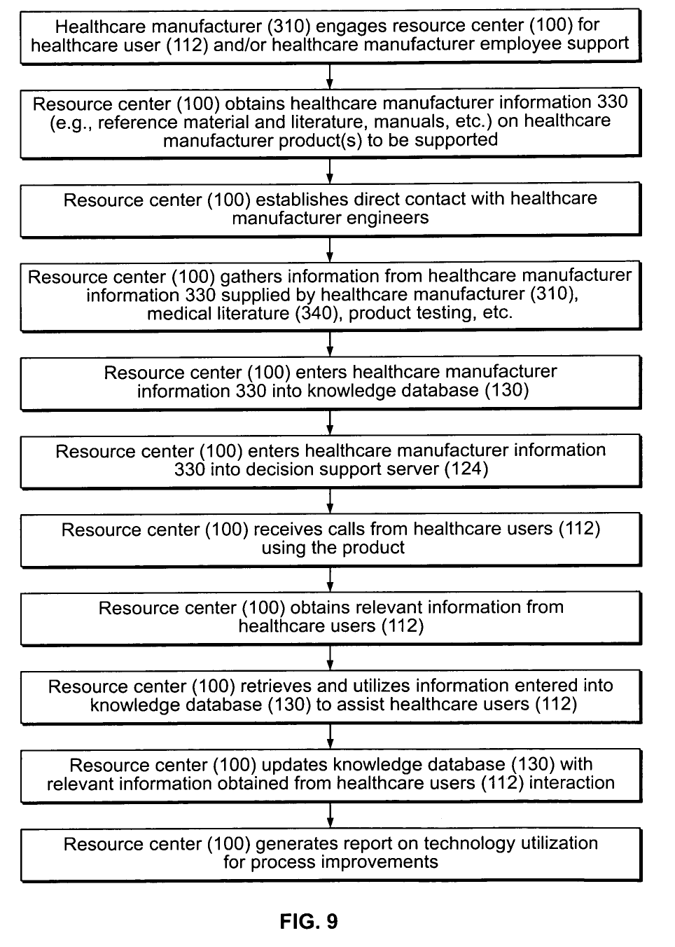

As seen in FIG. 9, also disclosed is a method for a healthcare manufacturer 310 to provide training and help services to a healthcare provider 120 or healthcare manufacturer employees comprising the step of establishing a connection between a clinical expert 138 and the healthcare provider 120 or healthcare manufacturer employee, wherein the clinical expert 138 provides education and training services.

As seen in FIG. 10, the remote assistance unit 101 preferably comprises a wearable headpiece or glasses 102, with a camera 103, microphone 104, headphone/headset 105 and HUD 106 utilizing input technologies such as wrist keyboard, finger mouse (e.g., of the type sold by Brando Workshop), voice activated command system, eye/head movement command system, or neural interface command system for example. Alternatively, a near retinal display could be used as a type of HUD 106. According to a preferred embodiment, the camera 103 is a remote controlled pan, tilt, zoom camera or an image capture camera. Voice communication would preferably use technologies such as VoIP.

The remote assistance unit 101 further comprises a cable 107 or a wireless personal area network (PAN) 108 (such as Bluetooth technology) to connect to a micro personal computer (MPC) 109 which has a mobile hot swappable power supply 111 that is connected to the MPC 109 by a power cable 113 or wireless power supply technology 115 (e.g., as disclosed in United States Published Patent Application 2006/0097667, the entire disclosure of which is herein incorporated by reference). The mobile hot swappable power supply 111 has the ability to provide mobile continuous power to the MPC 109. The system of the present invention permits the user to input data and navigate through websites or applications in a hands free manner such as head or eye movements or voice activation or with wearable input devices such as a finger mouse or wrist keyboard.

Preferably, the MPC 109 is sufficiently small in size such that it can be worn on the human body using a supportive garment. According to the present invention, the MPC 109 is worn using a customized garment or is imbedded within a garment. More preferably, the MPC 109 has the ability to launch software applications or a browser capable of accessing an intranet or internet. According to a further embodiment, the software application is capable of capturing images from real time video streams on either end of a bidirectional video image and storing the images or recording them for play back. Preferably, the software applications may also allow drawing and annotations to be placed on these images. More preferably, the software application comprises translational IVR (interactive voice response) software to provide multilingual aid to the user.

According to a preferred embodiment, the headphone/headset 105 and microphone 104 permit two way audio from the resource center 100 and the site requiring medical consulting services 240. According to another preferred embodiment, two way video capabilities are available on the HUD 106 to display the resource center 100 and a camera 103 to display the site requiring medical consulting services 240. According to another preferred embodiment, the system of the present invention further has the ability to project displayed images, e.g., with micro projector technology, to allow more than one individual to visualize information from the HUD 106. According to yet another preferred embodiment, the system of the present invention has the ability to display remote alarms, alerts, messages (including instant messages) or healthcare user data 320 to the healthcare user 112 and/or the healthcare provider 120. The system of present invention permits the healthcare user 112 and/or healthcare provider 120 to connect to the resource center 100 to display images, mpeg video conferences, electronic white boards (such as Adobe Flash Media Server) and/or other media for instruct/education or support.

The remote assistance unit 101 is preferably connected to a dedicated resource center 100 which has the needed expertise. The resource center 100 utilizes knowledge databases 130 and decision support databases 126 to provide assistance.

According to a preferred embodiment, data is transmitted from the MPC 109 to the resource center 100 through a cellular/3G connection 210 via a cellular tower 117. According to another preferred embodiment, the MPC 109 is connected to the resource center 100 through a wireless wide area network (WWAN) 119. In a further embodiment, the MPC 109 is connected to the resource center 100 through a wireless local area network (WLAN) (such as WiFi) by an access point 123 to the local area network (LAN) 125 through an external router 127 and an external firewall 129 to the Internet/wide area network (WAN) 110. In yet another embodiment, the MPC 109 sends data to the resource center 100 through a satellite connection 220 via a satellite transmitter 131. Preferably, a wireless method allows connection to the WLAN 121, WAN 110 and WWAN 119 through WiFi, or cellular/3g connection 210 or other technology that permits mobile connectivity.

Having now described a few embodiments of the invention, it should be apparent to those skilled in the art that the foregoing is merely illustrative and not limiting, having been presented by way of example only. Numerous modifications and other embodiments are within the scope of the invention and any equivalent thereto. It can be appreciated that variations to the present invention would be readily apparent to those skilled in the art, and the present invention is intended to include those alternatives.

Further, since numerous modifications will readily occur to those skilled in the art, it is not desired to limit the invention to the exact construction and operation illustrated and described, and accordingly, all suitable modifications and equivalents can be resorted to as falling within the scope of the invention.

What is claimed is:

1. A system comprising a resource center comprising:
a. a knowledge database,
b. a decision support server,
c. an interactive help system, and
d. a connection,
wherein the connection comprises a remote assistance unit.

2. The system of claim 1, wherein the resource center is a critical care resource center, a rapid response resource center, an emergency care resource center or mass casualty response center.

3. The system of claim 1, wherein the resource center is dedicated.

4. The system of claim 1, wherein the resource center is staffed 24/7.

5. The system of claim 1, wherein the resource center is accessible on demand.

6. The system of claim 1, wherein the resource center is staffed with clinical experts.

7. The system of claim 1, wherein the knowledge database comprises of at least one of healthcare manufacturer information, medical literature, reference material, a laboratory, clinical information or a clinical expert.

8. The system of claim 7, wherein the laboratory comprises a testing laboratory or an analysis laboratory.

9. The system of claim 1, wherein the decision support server comprises a software application.

10. The system of claim 1, wherein the interactive help system comprises an interface to a healthcare provider, a healthcare manufacturer or healthcare user.

11. The system of claim 10, wherein the healthcare manufacturer comprises at least one of a medical device manufacturer or a drug manufacturer.

12. The system of claim 1, wherein the interactive help system comprises at least one of an automated phone system, an intranet system, an internet access or a product provider help menu.

13. The system of claim 1, wherein the resource center further comprises a feedback loop.

14. The system of claim 13, wherein the feedback loop provides feedback to a healthcare manufacturer.

15. The system of claim 13, wherein the feedback loop provides feedback to the knowledge database.

16. The system of claim 1, wherein the resource center further comprises healthcare user data.

17. The system of claim 16, wherein the healthcare user data comprises at least one of demographic data, pharmacological data, physiological data, radiological images, hemodynamic parameters, laboratory data, device output data, audio data or video data.

18. The system of claim 1, wherein the remote assistance unit is portable.

19. The system of claim 1, wherein the remote assistance unit is wearable.

20. The system of claim 1, wherein the remote assistance unit comprises at least one of a heads-up display, a mini personal computer, a wireless local area network, a headset, a camera, voice over internet protocol technology, a microphone, glasses or head gear.

21. The system of claim 20, wherein the remote assistance unit further comprises retinal display technology.

22. The system of claim 20, wherein the remote assistance unit further comprises a wireless personal area network.

23. The system of claim 20, wherein the mini personal computer comprises input technologies.

24. The system of claim 20, wherein the mini personal computer comprises a wireless power supply.

25. The system of claim 20, wherein the camera is a remote controlled pan, tilt, zoom camera or an image capture camera.

26. The system of claim 20, wherein the remote assistance center further comprises projection technology.

27. The system of claim 20, wherein the remote assistance center further comprises instant messaging technology.

28. The system of claim 20, wherein the remote assistance center further comprises an electronic white board.

29. A method for providing training and help services to a healthcare provider, a healthcare manufacturer or a healthcare user, comprising the steps of
a. providing a resource center that comprises a knowledge database, a decision support server and an interactive help system,
b. building the knowledge database by compiling information,
c. analyzing the information in the knowledge database to develop the decision support server,
d. establishing a connection between the interactive help system and the healthcare provider, healthcare manufacturer and healthcare user,
e. accessing the decision support server, and
f. delivering education, training or consultation services via the interactive help system, wherein the connection comprises a remote assistance unit.

30. The method of claim 29, further comprising the step of providing a feedback loop.

31. The method of claim 30, wherein the step of proving feedback further comprises the step of providing feedback to the healthcare manufacturer via the feedback loop.

32. The method of claim 30, wherein the step of providing feedback further comprises the step of providing feedback to the knowledge database via the feedback loop.

33. The method of claim 29, further comprising the step of building the knowledge database by compiling information from at least one of healthcare manufacturer information, medical literature, reference material, a laboratory, clinical information or an expert clinical consultant.

34. The method of claim 33, further comprising the step of validating the information.

35. The method of claim 29, wherein the delivering step further comprises the step of delivering education, training and consultation services 24/7.

36. The method of claim 29, further comprising the step of collecting healthcare user data.

37. A method for healthcare manufacturers to provide training and help services to healthcare providers or healthcare manufacturer employees comprising the step of establishing a connection between a medical expert and the healthcare provider or healthcare manufacturer employee, wherein the connection comprises a remote assistance unit and the medical expert provides education and training services.
